(19) United States
(12) Patent Application Publication
(10) Pub. No.: US 2024/0115751 A1
(43) Pub. Date: Apr. 11, 2024

(54) SUBSTRATE FOR DIFFUSING VOLATILE SUBSTANCES

(71) Applicant: ZOBELE HOLDING SPA, Trento (IT)

(72) Inventors: Sergio LUQUE VERA, BARCELONA (ES); Alberto Jose SANCHO MARZO, BARCELONA (ES); Maria GARCIA SUBIRATS, BARCELONA (ES); David ALONSO CANADAS, BARCELONA (ES)

(21) Appl. No.: 18/270,177

(22) PCT Filed: Dec. 22, 2021
(86) PCT No.: PCT/EP2021/087433

§ 371 Date: Jun. 28, 2023

(30) Foreign Application Priority Data
Dec. 28, 2020 (ES) P202031304

(51) Int. Cl. A61L 9/12

(52) U.S. Cl. CPC A61L 9/12

(57) ABSTRACT

The substrate for diffusing volatile substances comprises a dispersible binder and a natural material, and the percentages by weight in the dry state are: dispersible binder: 15-45%; and natural material: 55-75%. It may also comprise a preservative. It provides a substrate that provides constant and durable air diffusion properties.

The present invention relates to a substrate for diffusing volatile substances, which provides constant and long-lasting diffusion properties in air.

### BACKGROUND OF THE INVENTION

In air diffuser applications, the use of substrates containing a certain amount of volatile liquid substance and ensuring transport to an evaporation area is known.

The substrate must be able to hold a relatively large amount of liquid and therefore occupy a large volume. The common application requires a duration of at least 15 days of refill duration, which translates into storage volumes in the order of 1 to 10 ml.

In natural substrate materials, a high liquid storage capacity requires a large volume and thus a large surface area exposed to air. Since the evaporation rate is directly proportional to the evaporation area, a high volume also increases the evaporation rate and thus reduces the refill duration.

In addition, in solid substrate materials, the evaporation rate is proportional to the amount of liquid transported to the evaporation area (capillary properties), which change according to the amount of liquid contained in the substrate matrix. When the substrate is highly saturated (new refill), the transport and evaporation of liquid into the air is higher compared to low saturated conditions (end of life).

### DESCRIPTION OF THE INVENTION

Therefore, there is a need for a substrate that offers both high storage capacity and a constant evaporation rate during its lifetime by low and constant capillarity properties.

Therefore, one purpose of the present invention is to provide a substrate that provides constant and durable air diffusion properties.

With the substrate according to the present invention it is possible to solve said drawbacks, providing other advantages which are described below.

The substrate for diffusing volatile substances according to the present invention comprises a mixture of different natural ingredients with different granularities and a dispersible binder.

The substrate is made of natural particles and a dispersible binder material, such as flour-like materials, mixed with different granular grade materials, such as wood, e.g. sawdust. The composition is processed with water by conventional processes such as, for example, compression molding or extrusion. After processing, the substrate is dried to a solid state.

According to claim 1, the substrate for the diffusion of volatile substances according to the present invention comprises:
a dispersible binder; and
a natural material.

In addition, it may also comprise a preservative.

Advantageously, the percentages by weight in the dry state are as follows:
dispersible binder: 15-45%;
natural material: 55-75%; and
preservative: 0.1-5%.

More preferably, the percentages by weight in the dry state are as follows:
dispersible binder: 20-30%;
natural material: 65-75%; and
preservative: 0.5-1.5%.

According to a preferred embodiment, the granulometry of the dispersible binder is comprised between 100 and 250 μm, such as 180 μm, and the granulometry of the natural material is between 0 and 250 μm, such as less than 60 μm.

Preferably, the dispersible binder is flour and the natural material is wood, such as wood sawdust.

In addition, the preservative is preferably sodium benzoate.

The substrate provides a high storage capacity and a constant low capillarity, which ensures a long refill life at a constant evaporation rate.

The encapsulation of the substance within a granular matrix hinders the transport through the substrate to the evaporation area. This results in low capillary properties, which in turn also favors a lower gradient of substance between different areas of the substrate. Both effects result in a constant low supply of substance to the evaporation area and thus a constant and long-lasting evaporation rate.

### DESCRIPTION OF A PREFERRED EMBODIMENT

As an example, the substrate according to the present invention has the following composition.

| | Granulometry (μm) | |
|---|---|---|
| Substrate composition | Interval | Preferential |
| Dispersible binder (flour) | 100-250 | 180 |
| Natural material (wood) | 0-250 | <60 |
| Preservative | — | — |
| Water | — | — |

| | % by weight, | | | |
|---|---|---|---|---|
| | hydrated | | % by weight, dry | |
| Substrate composition | Interval | Preferential | Interval | Preferential |
| Dispersible binder (flour) | 5-15% | 7-10% | 15-45% | 20-30% |
| Natural material (wood) | 15-30% | 20-25% | 55-75% | 65-75% |
| Preservative | 0.1-5% | 0.5-1% | 0.1-5% | 0.5-1.5% |
| Water | 40-80% | 50-70% | — | — |

| | Grain size range | | % by weight, | | % by weight, | |
|---|---|---|---|---|---|---|
| | (μm) | | hydrated | | dry | |
| Substrate | | Prefer- | | Prefer- | | Prefer- |
| composition | Interval | ential | Interval | ential | Interval | ential |
| Dispersible | 100-250 | 180 | 5-15% | 7-10% | 15-45% | 20-30% |
| binder (flour) | | | | | | |
| Natural | 0-250 | <60 | 15-30% | 20-25% | 55-75% | 65-75% |
| material | | | | | | |
| (wood) | | | | | | |
| Preservative | — | — | 0.1-5% | 0.5-1% | 0.1-5% | 0.5-1.5% |
| Water | — | — | 40-80% | 50-70% | — | — |

Furthermore, according to a preferred embodiment, the substrate according to the present invention is disc-shaped, although it may have a different shape, and its thickness changes from 0.1 to 20 mm.

A non-limiting example of substrate composition is:
Dispersible binder (flour): 25.8% by weight on dry condition
Natural material (wood): 73.2% by weight in dry condition
Preservative (sodium benzoate): 1% by weight on dry condition

Although reference has been made to a specific embodiment of the invention, it is obvious to a person skilled in the art that the substrate described herein is susceptible to numerous variations and modifications, and that all the details mentioned may be replaced by technically equivalent ones without departing from the scope of protection defined by the appended claims.

1. A substrate for diffusing volatile substances, characterized in that the substrate comprises:
a dispersible binder; and
a natural material.

2. The substrate for diffusing volatile substances according to claim 1, wherein the percentages by weight in the dry state are as follows:
dispersible binder: 15-45%; and
natural material: 55-75%.

3. The substrate for diffusing volatile substances according to claim 1, wherein the percentages by weight in the dry state are as follows:
dispersible binder: 20-30%; and
natural material: 65-75%.

4. The substrate for diffusing volatile substances according to claim 1, further comprising a preservative.

5. The substrate for diffusing volatile substances according to claim 4, wherein the percentage by weight in dry state of the preservative is between 0.1 and 5%, in particular between 0.5 and 1.5%.

6. The substrate for diffusing volatile substances according to claim 1, wherein the granulometry of the dispersible binder is between 100 and 250 μm.

7. The substrate for diffusing volatile substances according to claim 6, wherein the granulometry of the dispersible binder is 180 μm.

8. The substrate for diffusing volatile substances according to claim 1, wherein the granulometry of the natural material is between 0 and 250 μm.

9. The substrate for diffusing volatile substances according to claim 8, wherein the granulometry of the natural material is less than 60 μm.

10. The substrate for diffusing volatile substances according to claim 1, wherein the dispersible binder is flour.

11. The substrate for diffusing volatile substances according to claim 1, wherein the natural material is wood.

12. The substrate for diffusing volatile substances according to claim 11, wherein the natural material is wood sawdust.

13. The substrate for diffusing volatile substances according to claim 4, wherein the preservative is sodium benzoate.

14. The substrate for diffusing volatile substances according to claim 2, further comprising a preservative.

15. The substrate for diffusing volatile substances according to claim 3, further comprising a preservative.

16. The substrate for diffusing volatile substances according to claim 2, wherein the granulometry of the dispersible binder is between 100 and 250 μm.

17. The substrate for diffusing volatile substances according to claim 3, wherein the granulometry of the dispersible binder is between 100 and 250 μm.

18. The substrate for diffusing volatile substances according claim 2, wherein the granulometry of the natural material is between 0 and 250 μm.

19. The substrate for diffusing volatile substances according claim 3, wherein the granulometry of the natural material is between 0 and 250 μm.

20. The substrate for diffusing volatile substances according to claim 5, wherein the preservative is sodium benzoate.
